(19) United States
(12) Patent Application Publication
(10) Pub. No.: US 2016/0256636 A1
(43) Pub. Date: Sep. 8, 2016

(54) SKIN PIERCING TOOL FOR LOCALLY PIERCING HUMAN OR ANIMAL SKIN AND A PIERCING TOOL ARRANGEMENT

(71) Applicant: MT.DERM GmbH, Berlin (DE)

(72) Inventor: Dirk Scherkowski, Berlin (DE)

(21) Appl. No.: 15/059,611

(22) Filed: Mar. 3, 2016

(30) Foreign Application Priority Data
Mar. 6, 2015 (EP) 15 158 064.4

(51) Int. Cl. A61M 5/32

(52) U.S. Cl. CPC A61M 5/3297

(57) ABSTRACT

The disclosure refers to a skin piercing tool for locally piercing human or animal skin, comprising a proximal tool section; a distal tool section, in which a piercing tip is arranged; one or more needles, in which a needle shaft is provided in the area of the proximal tool sections and a needle tip is provided in the area of the distal tool section as part of the piercing tip; and a supply reservoir that extends as an open reservoir between opposing needle sections of the one or more needles and extends toward the proximal tool section behind the piercing tip, wherein the supply reservoir is in fluid communication with the piercing tip.

### CROSS-REFERENCE TO RELATED APPLICATIONS

The present application claims priority under 35 U.S.C. §119 to European Patent Application No. 15 158 064.4, filed Mar. 6, 2015, which is hereby incorporated by reference in its entirety.

The present disclosure relates to a skin piercing tool for locally piercing human or animal skin, and a piercing tool arrangement.

### BACKGROUND OF THE INVENTION

Skin piercing tools are used to pierce human or animal skin locally, for example in order to introduce a substance into the skin. If a substance is to be deposited, it may be for example an ink, a medicinal substance, and/or a cosmetic substance. If the substance is ink, it may be used to apply a tattoo or permanent make-up.

Skin piercing tools may consist of one or more needles. If multiple needs are involved, a needle group may be formed. Thus, the document US 2012/0029549 A1 discloses a tattoo needle device in which multiple single needles are combined to form a group of needles, in which needle shafts are soldered to each other. Starting from the lengthwise direction of the respective needle in the area of the needle shaft, the needles are curved inwards in the area with the needle tip, so that piercing tips of the needles can be arranged in close fitting manner side by side.

Document JP 2000342332 A also describes skin piercing tools in which multiple needles are combined to create a needle group. More piercing tools comprising multiple needles are described in the document CN 201862140 U. Single needles and piercing devices in which multiple needles are arranged in the area of an application surface (needle plate) are disclosed in document US 2011/0042847 A1.

Other needles for puncturing skin are described in the document US 2005/0066775 A1.

A skin piercing tool is known from document US 2007/0299388 A1, in which multiple projections are provided over a flat surface, each such projection having a piercing tip. In one embodiment, microprojections arranged adjacent to each other may also be inclined toward each other, so that a supply reservoir is formed between the inclined microprojections, in which reservoir a liquid to be introduced into the skin may collect.

A skin piercing tool is known from document U.S. Pat. No. 768,413, in which a piercing needle is extended and retracted through an opening in a housing. Inside the housing, a leaf spring is arranged with which pressure can be exerted on the piercing needle from the side, wherein the force of the pressure applied may be varied by means of an adjusting screw.

### SUMMARY OF THE INVENTION

It is an object of the present disclosure to provide a skin piercing tool for locally piercing human or animal skin, and a piercing tool arrangement comprising multiple skin piercing tools, which enable more efficient local piercing of the skin.

In order to solve this object, a skin piercing tool according to claim 1 for locally piercing human or animal skin is created. A piercing tool arrangement comprising multiple skin piercing tools according to independent claim 14 is also provided. Variations are the subject matter of the dependent claims.

According to one aspect, a skin piercing tool is provided for locally piercing human or animal skin. The skin piercing tool has a proximal tool section and a distal tool section, in which a piercing tip is arranged, which tip is used when the skin piercing tool is applied to or against the skin surface. One or more needles are provided, in which the needle shafts are arranged in the area of the proximal tool section, and a needle tip is arranged as part of the piercing tip in the area of the distal tool section. A supply reservoir is formed, extending as an open reservoir between opposing needle sections of the one or multiple needles in the direction of the proximal tool section behind the piercing tip. The supply reservoir is in fluid communication with the piercing tip. The oppositely positioned needle sections of the needle, or on one of the multiple needles adjacent to the supply reservoir, have a needle section, that is inclined or curved away from the lengthwise axis of the needle, and another needle section that is arranged between the needle section and the needle tip, and is inclined or curved toward the lengthwise axis of the needle.

According to a further aspect, a piercing tool arrangement having multiple skin piercing tools is provided for locally piercing human or animal skin, wherein the multiple skin piercing tools are arranged in a shared tool holder.

The supply reservoir is in fluid communication with the piercing tip, in such manner that during operation a substance from the supply reservoir is able to flow as far as the piercing tip.

The oppositely positioned needle sections may also be referred to as opposing needle sections. The opposing needle sections may be provided as needle sections facing each other.

In the skin piercing tool, the needle section that is inclined or curved away from the lengthwise axis of the needle and the other needle section, which is inclined or curved toward the lengthwise axis of the needle, are arranged adjacent to the supply reservoir and to this extent are designed to form or define the borders of the open reservoir.

The inclined needle sections may be straight.

Various needle types may be used, including for example flat needs, which may be stamped from a steel sheet, for example, as well as solid and hollow needles, which may have a round, oval or angular cross section. A single needle may also have different cross sectional shapes in various sections in the lengthwise direction.

The skin piercing tool or piercing tool arrangement with multiple skin piercing tools may be mounted detachably or non-detachably on a handheld unit for local piercing of a human or animal skin. In one variant, the handheld unit may have a driving device, with which the skin piercing tool or piercing tool arrangement is set in forward or backward motion. In one variant, the driving device converts electrical energy to produce a driving force for moving the skin piercing tool or piercing tool arrangement. Handheld units of such kind are known per se in various embodiments, particularly with non-manual drive.

The piercing tip may be made from multiple needle tips. The needle sections which culminate in the needle tip of the respective needled may be designed identically or differently, particularly in terms of their shape with regard to the cross section of the needle. The alignment of each needle section with the needle tip relative to the lengthwise axis of the needle may be identical or different. The needle sections with the respective needle tip may extend at an angle or parallel to the lengthwise axis of the needle in the direction of the piercing tip.

At least some of the needle tips may be arranged one behind the other along a skin piercing or puncturing direction. The skin piercing or puncturing direction may be designed to be essentially parallel to the lengthwise axis of the needle. The at least some of the multiple needle tips that are arranged to lie one behind the other along the piercing direction lie on a puncturing line of the skin piercing tool.

The supply reservoir may be open towards a separation zone between the needle tips that are arranged one behind the other.

At least some of the multiple needle tips may be arranged side by side in a plane transverse to the lengthwise axis. The needle tips that lie side by side in a plane transverse to the lengthwise axis may be at a distance from each other, or they may touch each other. The multiple needle tips may be formed on the one needle. Alternatively, the multiple needle tips may be formed on multiple needles.

The respective lengthwise axes of the multiple needle tips may be arranged at an angle that is not zero with respect to each other. The lengthwise axis of the needle tip corresponds to the axial direction in the needle section of which the back is directly adjacent to the needle tip.

A bending or springy section may be formed in an area of the needle between the needle section and the next needle section, such that the needle bends in springy manner when pressure is applied to the associated needle tip. The springy bending movement may cause the open reservoir to change its shape and/or size momentarily.

A direct transition may be formed between the needle section and the next needle section. The next needle section may be immediately and directly adjacent to the needle section. The transition between the two needle sections may be formed for example by the bending or springy section. The direct transition have the form of a curved transition or an angular transition.

A straight needle section may be arranged in the area of the supply reservoir opposite the needle section and/or the next needle section. The straight needle section may be a part of the needle section that defines and borders the open reservoir. Needle shafts of the multiple needles may form a common tool shaft. The needle shafts may be twisted and/or soldered together. The needle shafts may be arranged on a common shaft mounting, to form a needle plate, for example, in which multiple needles or multiple skin piercing tools are arranged over an application surface. The needle shafts may be located at a distance from each other and they may be directly touching each other.

One or more side openings in the open reservoirs may each be in the form of a slot.

The next needle section, which is inclined or curved toward the lengthwise axis of the needle, may end in a needle tip.

The variations explained with regard to the skin piercing tool may be provided for one or more of the skin piercing tools on the piercing tool arrangement. The multiple skin piercing tools may be of identical or different design. The piercing tips of the respective skin piercing tools may form a common piercing tip in the piercing tool arrangement, wherein the piercing tips may be arranged in a common plane or they may be offset in planes aligned one behind the other.

### DESCRIPTION OF THE DRAWING FIGURES

In the following, further embodiments are described with reference to figures of a drawing. In the drawing:

FIG. 1 is a schematic representation of multiple skin piercing tools in which needle sections are curved,

FIG. 2 is a schematic representation of multiple skin piercing tools in which straight needle sections are inclined or angled away from the lengthwise axis of the needle and toward the lengthwise axis of the needle,

FIG. 3 is a schematic representation of multiple skin piercing tools in which needles have needles sections that are each straight and are inclined or angled toward the lengthwise axis of the needle,

FIG. 4 is a schematic representation of skin piercing tools according to a further embodiment,

FIG. 5 is a schematic representation of further skin piercing tools,

FIG. 6 is a schematic representation of other skin piercing tools,

FIG. 7 is a schematic representation of a skin piercing tool with a flat needle,

FIG. 8 is a schematic representation of skin piercing tools, each of which comprises more than two needles,

FIG. 9 is a schematic representation of a piercing tip of a skin piercing tool, in which needle tips are arranged one behind the other, and

FIG. 10 is a schematic representation of piercing tips of a skin piercing tool, in which needle tips are arranged flush beside each other.

### DESCRIPTION OF CERTAIN EMBODIMENTS OF THE INVENTION

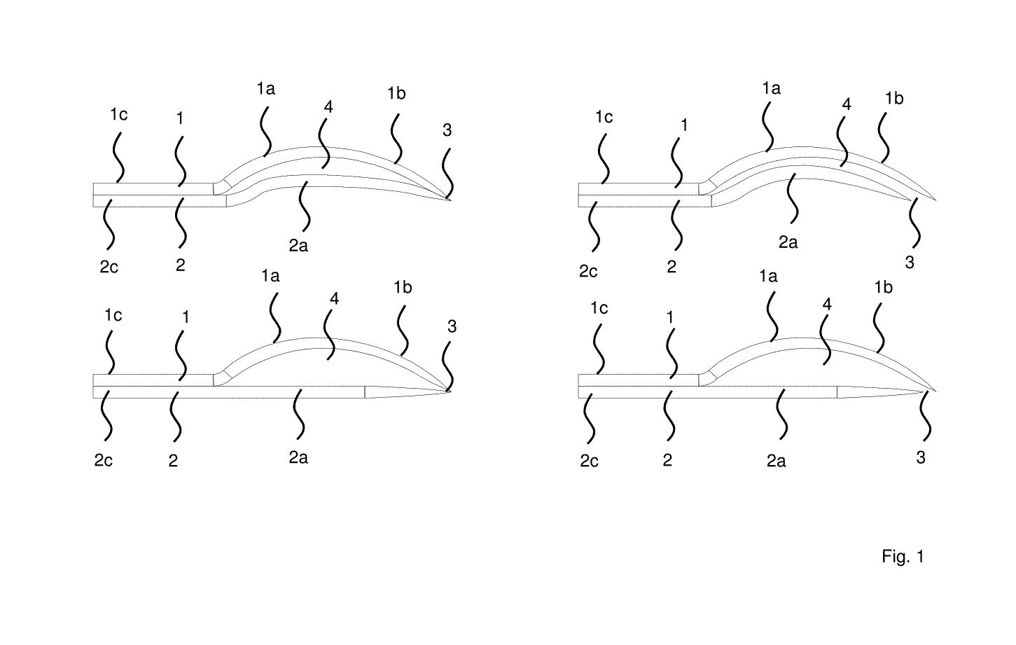

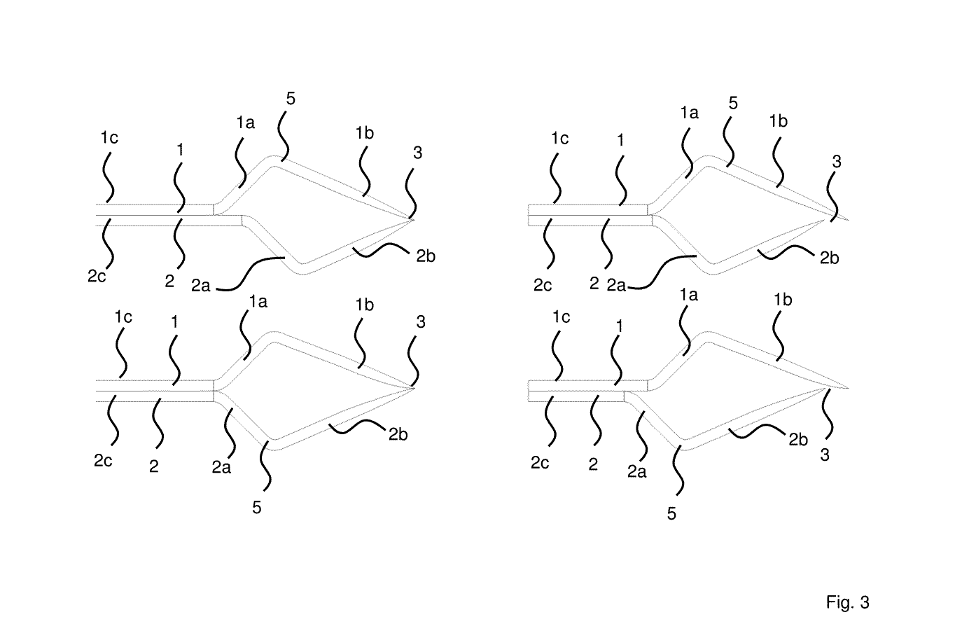

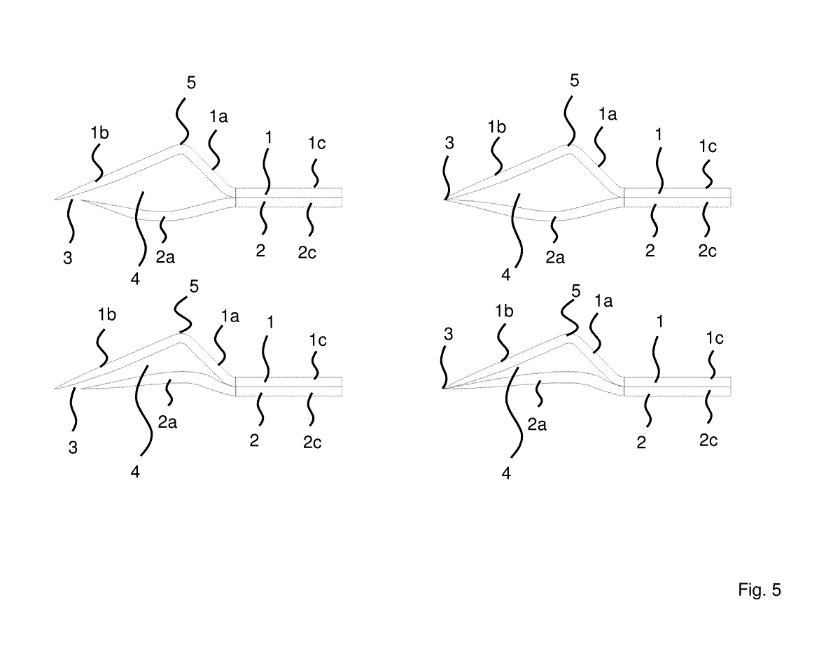

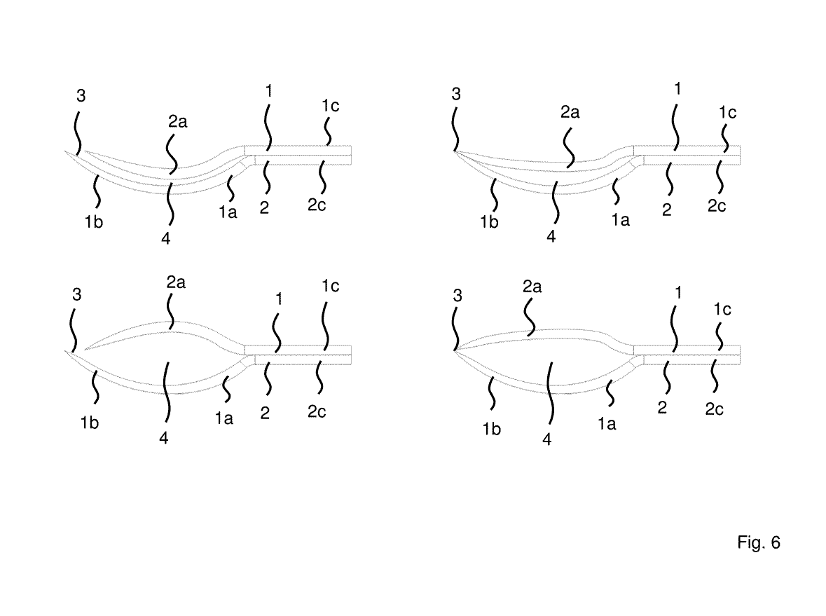

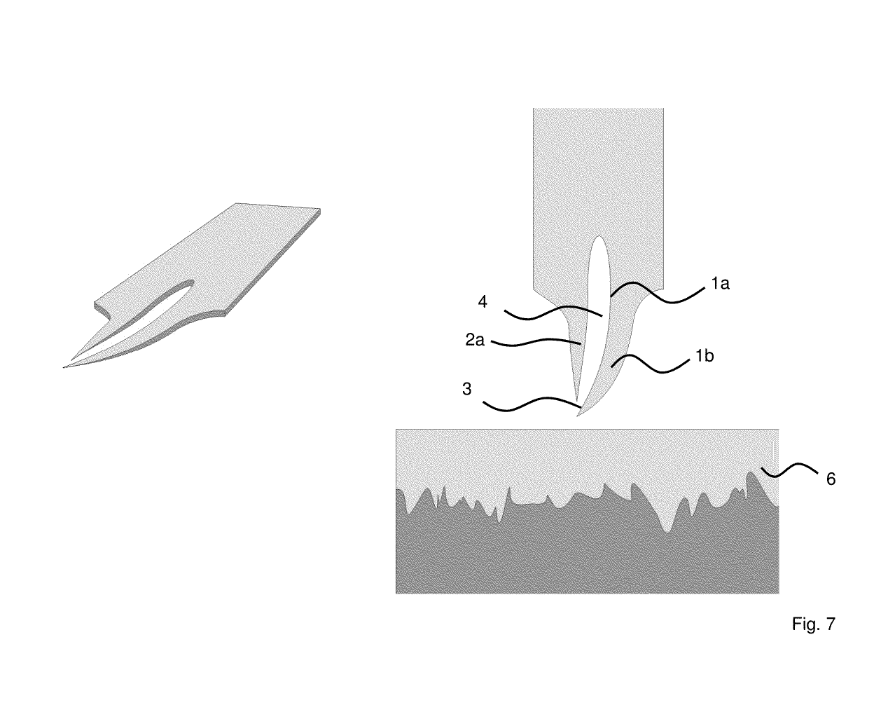

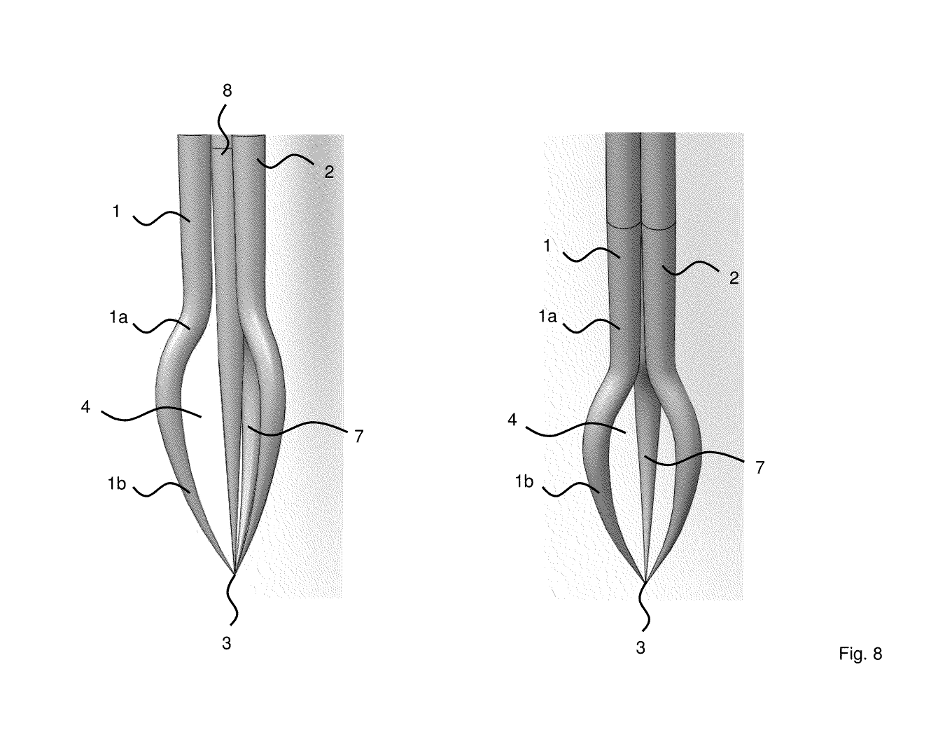

In the following, embodiments of a skin piercing tool for locally piercing human or animal skin will be explained with reference to FIGS. 1 to 8. In the embodiments shown in FIGS. 1 to 4, each skin piercing tool has a first and a second needle 1, 2. In FIGS. 5 and 6, further variations of skin piercing tools are shown. In the embodiment in FIG. 7, a flat needle is used. FIG. 8 shows a variant of a skin piercing tool having more than two needles.

All skin piercing tools have a piercing tip 3, which is made up of at least two needle tips. In this context, both needle tips may be arranged one behind the other in a piercing direction, or in a converging pattern to form a common piercing tip. The piercing direction may extend parallel to the lengthwise axis of the skin piercing tool.

In the variants shown in FIG. 1, a is bent (curved) needle section la is aligned or inclined away from the lengthwise axis of the needle. In comparison, a bent (curved) needle section lb adjacent thereto is also aligned or inclined toward the lengthwise axis of the needle. A needle section 2a in the second needle 2 opposite the section in the first needle is either straight or curved. A supply reservoir 4 designed as an open reservoir is formed between needle sections la, lb of the first needle 1 and needle section 2a of the second needle 2. A substance intended for introduction in the to skin may be collected in supply reservoir 4, so that it may then flow to piercing tip 3 during operation. In the embodiment shown, needle shafts 1c, 2c of the two needles 1, 2 are arranged side by side.

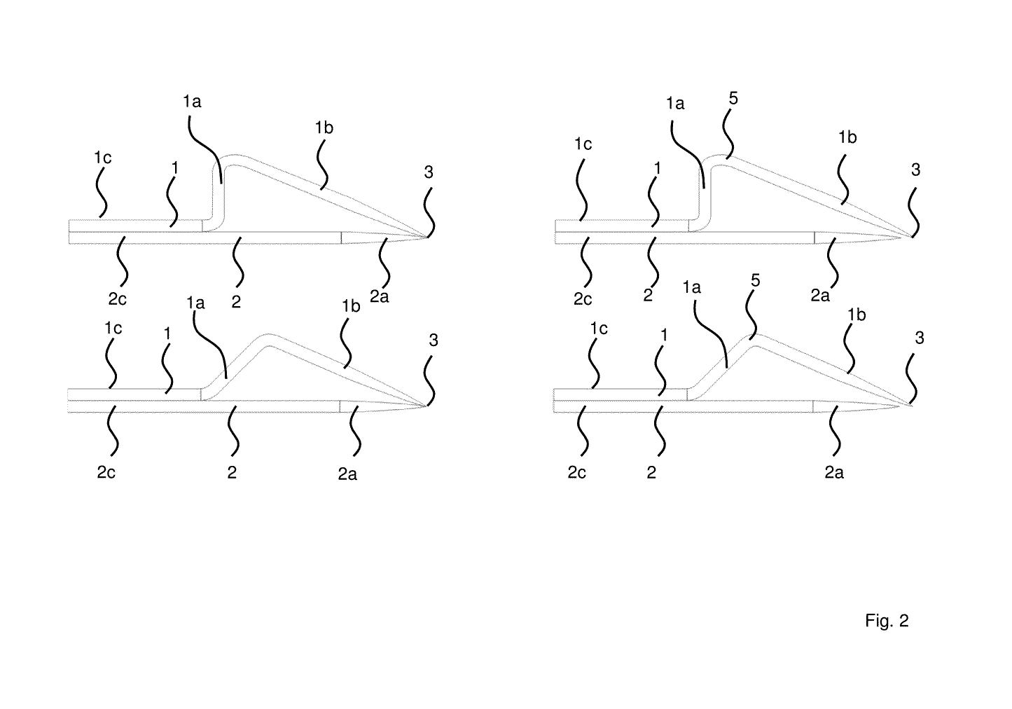

In the embodiments of FIG. 2, the needle section 1 a inclined or aligned away from the lengthwise axis of the needle and the needle section lb that is inclined or aligned toward the lengthwise axis of the needle are designed as straight needle sections. Between these needle sections, a corner or bend area 5 is arranged, at which the two needle sections la, lb converge with each other, particularly in such manner that the assigned needle bends in springy manner when pressure is applied to the piercing tip.

For comparison, the skin piercing tool in the variants of 3 has straight needle sections 2a, 2b for the second needle 2.

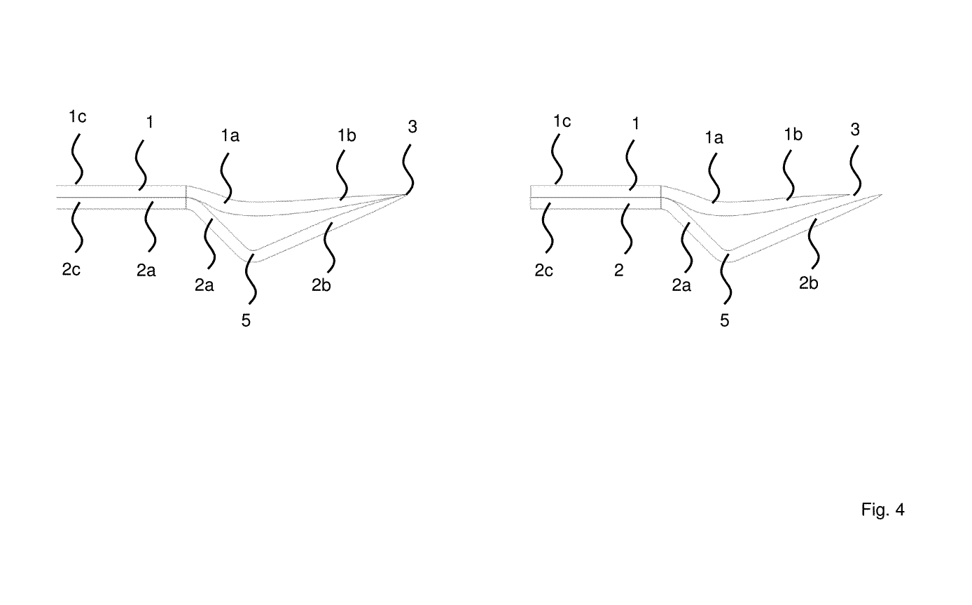

In the skin piercing tool according to the variants of FIG. 4, curved and straight needle sections are used in combination in the area of supply reservoir 4.

FIGS. 5 and 6 show further variants of skin piercing tools.

FIG. 7 shows schematic representations of a flat needle with supply reservoir 4, the needle tips are created on the same flat needle, which is made from a sheet steel material, for example. The flat needle in FIG. 7 is shown above a skin surface 6 which is shown in cross section. The section la that is aligned or inclined away from the lengthwise axis of the needle is bent (curved) in a section adjacent to the supply reservoir 4, as is the needle section lb adjacent thereto, which is again aligned or inclined toward the lengthwise axis of the needle. The opposite needle section 2a, which may be straight or curved, at least on the side thereof facing the supply reservoir 4, is formed on the same flat needle. Alternatively, one or both opposing and separated needle sections may provided on different needles, particularly in a section distal to the piercing tip 3.

FIG. 8 shows an embodiment of a skin piercing tool having a total of four needles 1, 2, 7, 8 and a variant with three needles 1, 2, 7.

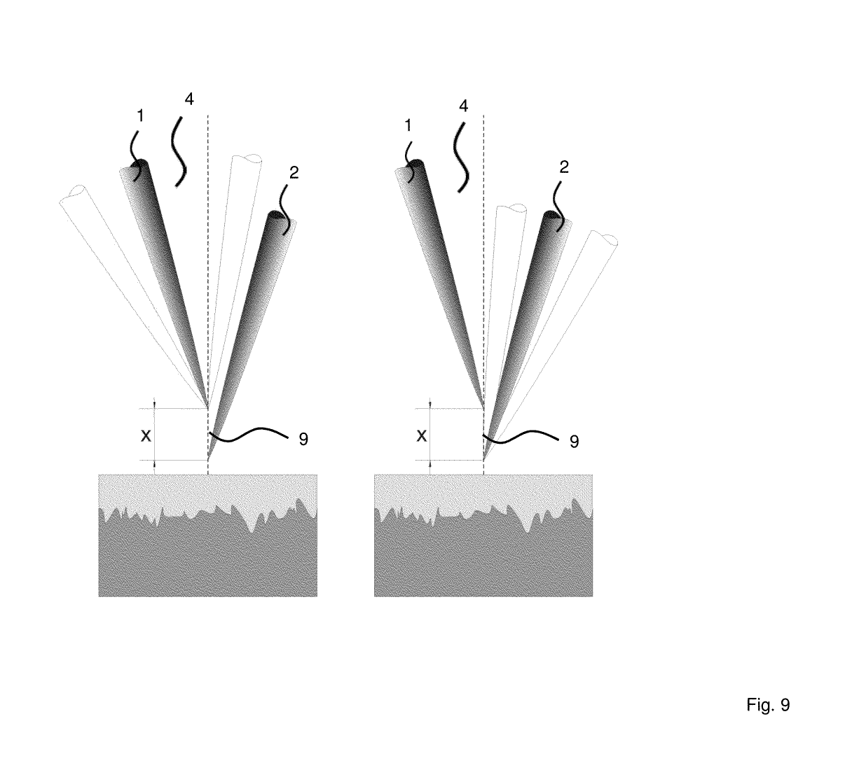

FIG. 9 shows schematic representations of piercing tip 3 for different variations, in which the needle tips are arranged one behind the other. The supply reservoir 4 located behind piercing tip 3 opens into a space 9 between the needle tips arranged one behind the other, which space has a length x in the lengthwise direction of the needle.

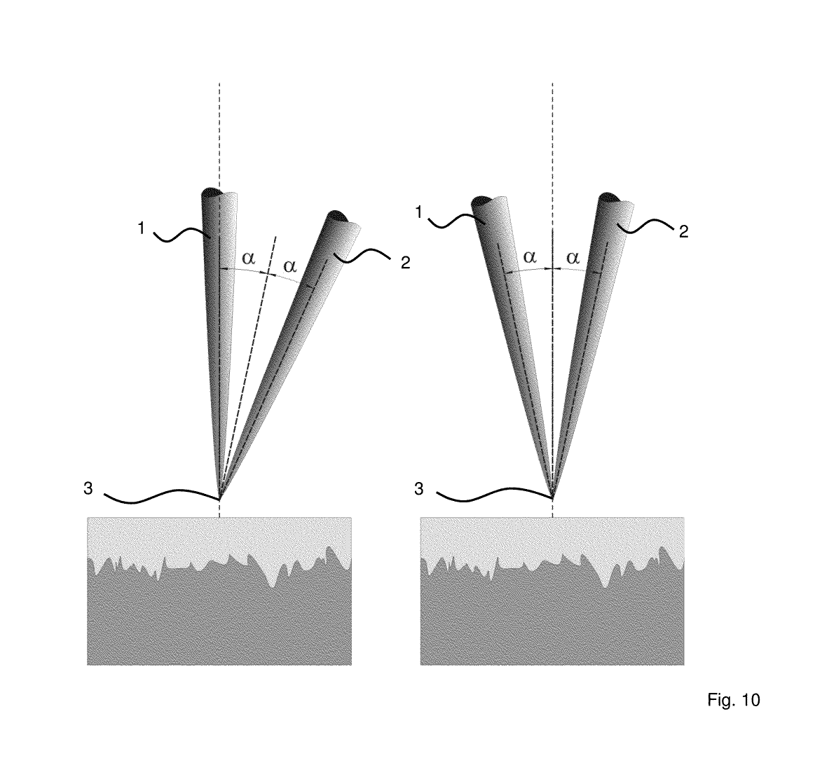

In contrast to this, the needle tips in the embodiments of piercing tip 3 shown in FIG. 10 are arranged so that they touch.

The features disclosed in the preceding description, and in the claims and the drawing may be significant for the realisation of the different variations either individually or in any combination thereof.

1. Skin piercing tool for locally piercing human or animal skin, comprising a proximal tool section;
a distal tool section, in which a piercing tip is arranged;
one or more needles, in which a needle shaft is provided in the area of the proximal tool sections and a needle tip is provided in the area of the distal tool section as part of the piercing tip; and
a supply reservoir that extends as an open reservoir between opposing needle sections of the one or more needles and extends toward the proximal tool section behind the piercing tip, wherein the supply reservoir is in fluid communication with the piercing tip;
wherein
the opposing needle sections on the one needle or on one of the multiple needles have a needle section that is provided adjacent to the supply reservoir and inclined or curved toward the lengthwise axis of the needle;
the opposing needle sections on the one needle or on one of the multiple needles have a further needle section that is provided adjacent to the supply reservoir and inclined or curved away from the lengthwise axis of the needle; and
the needle section is arranged between the further needle section and the needle tip.

2. Skin piercing tool according to claim 1, wherein the piercing tip is formed with multiple needle tips.

3. Skin piercing tool according to claim 2, wherein at least some of the multiple needle tips are arranged one behind the other along a piercing direction.

4. Skin piercing tool according to claim 3, wherein the supply reservoir is open toward a space between the needle tips that are arranged one behind the other.

5. Skin piercing tool according to claim 2, wherein at least some of the multiple needle tips are arranged side by side in a plane transverse to the lengthwise axis.

6. Skin piercing tool according to claim 2, wherein the multiple needle tips are formed on the needle.

7. Skin piercing tool according to claim 2, wherein respective lengthwise axes of the multiple needle tips are arranged at an angle other than zero relative to each other.

8. Skin piercing tool according to claim 1, wherein a bending or springy section is provided in an area of the needle between the needle section and the next needle section, such that the needle bends in springy manner when pressure is applied to the associated needle tip.

9. Skin piercing tool according to claim 1, wherein a direct transition is created between the needle section and the next needle section.

10. Skin piercing tool according to claim 1, wherein a straight needle section is arranged opposite the needle section and/or the next needle section in the area of the supply reservoir.

11. Skin piercing tool according to claim 1, wherein needle shafts of the multiple needle together form a common tool shaft.

12. Skin piercing tool according to claim 1, wherein one or more side openings in the open reservoir are realised individually as slots.

13. Skin piercing tool according to claim 1, wherein the end of the next needle section, which is inclined or curved toward the lengthwise axis of the needle, terminates in a needle tip.

14. A piercing tool arrangement comprising multiple skin piercing tools for locally piercing human or animal skin arranged in a shared tool holder, at least one of the skin piercing tools comprising:
a proximal tool section;
a distal tool section, in which a piercing tip is arranged;
one or more needles, in which a needle shaft is provided in the area of the proximal tool sections and a needle tip is provided in the area of the distal tool section as part of the piercing tip; and
a supply reservoir that extends as an open reservoir between opposing needle sections of the one or more needles and extends toward the proximal tool section behind the piercing tip, wherein the supply reservoir is in fluid communication with the piercing tip;
wherein
the opposing needle sections on the one needle or on one of the multiple needles have a needle section that is provided adjacent to the supply reservoir and inclined or curved toward the lengthwise axis of the needle;
the opposing needle sections on the one needle or on one of the multiple needles have a further needle section that is provided adjacent to the supply reservoir and inclined or curved away from the lengthwise axis of the needle; and
the needle section is arranged between the further needle section and the needle tip.
